(19) United States
(12) Patent Application Publication
(10) Pub. No.: US 2016/0296712 A1
(43) Pub. Date: Oct. 13, 2016

(54) A SAFETY CLIP

(71) Applicant: METIER MEDICAL LIMITED, Bundall (AU)

(72) Inventor: Larry MINIX, Ponte Vedra Beach, FL (US)

(21) Appl. No.: 15/101,350

(22) PCT Filed: Dec. 2, 2014
(86) PCT No.: PCT/AU2014/001090

§ 371 Date: Jun. 2, 2016

(30) Foreign Application Priority Data
Dec. 5, 2013 (AU) 2013904740

(51) Int. Cl. A61M 5/32

(52) U.S. Cl. CPC A61M 5/3216

(57) ABSTRACT

The present invention relates to a safety clip for needles. The present invention is adapted for Incorporation as a standalone item, or integrated with a needle, or with a needle and syringe. The standalone safety clip (10) is comprised of an attachment collar (20), elongate sheath (30) and hinge (40). The attachment collar (20) has a central aperture (60) for attaching the hub of a syringe or the collar of a needle base. Attachment collar (20) is further comprised of slots (60) which engage ramped tabs (70) of the elongate sheath, such that when inserted onto the hub of the syringe or collar of the base of the needle, the elongate sheath (30) is able to be pivoted from a first position In which the needle Is exposed and able to be used, and a second position in which at least, the needle tip is sheathed in a longitudinal channel (90) of the sheath (30) such that the user Is unable to be pricked by the needle, and wherein when placed into the second, sheathed position, the user is prevented from returning the elongate sheath to the firs!, exposed position by virtue of the ramped tabs (70) becoming locked into place by the slots (60).

### TECHNICAL FIELD

The field of the present invention lies in medical devices and particularly, safety devices to minimise the risk of needlestick injuries.

### BACKGROUND ART

Medical staff need to routinely administer pharmaceutical products or withdraw bodily fluids by way of a hypodermic needle inserted into the body of the patient. These staff must dispose of the used needles in a safe and effective manner. Whilst containers have been in use to accept used needles and syringes, the containers do not address the risks to the medical staff after the injection or withdrawal of fluids and before the needle and syringe are inserted into the container.

In order to address this problem, certain sheaths and/or safety clips have been devised to be attached to the needle and/or syringe. However these clips have suffered problems with reliability and have not been taken up by the medical sector. It is an object of the current invention to substantially ameliorate some of the deficiencies of the prior art.

### DISCLOSURE OF INVENTION

According to the present invention, there is provided a safety clip for a needle, the safety clip comprising:

an attachment collar having a central aperture through which can be secured either a hub of a syringe with the needle or a collar at the base of the needle,

an elongate sheath having a longitudinal channel along at least one side thereof for receiving the needle,

a hinge operably connected between the attachment collar and the elongate sheath,

wherein the attachment collar further comprises one or more slots containing retaining members, the slots being located at a remote end of the attachment collar, which remote end is furthest from an end of the attachment collar to which the hinge is connected,

and wherein the elongate sheath further comprises one or more mushroom shaped projections which extend from a remote end of the elongate sheath in a direction parallel to the longitudinal channel, and are insertable into the slots and engage with the retaining members,

such that the elongate sheath of the safety clip, when the safety clip is secured by its attachment collar to a hub of a syringe with the needle or to a collar at the base of the needle, can be pivoted about the hinge between a first position in which the needle is exposed and able to be used, and a second position in which at least a tip of the needle is sheathed in the longitudinal channel so that a user cannot be pricked by the needle,

and wherein the retaining members are shaped to prevent the mushroom shaped projections being removed from their engagement with the retaining members, thereby providing a permanent and secure locking between the slots and the mushroom shaped projections which prevents the elongate sheath being pivoted back to the first position when placed in the second position.

Preferably, the or each slot contains three retaining members.

In a preferred form, there are two slots and there are two mushroom shaped projections.

In a further preferred form, the or each mushroom shaped projection has a recess and a ramped portion which has a thickest point at which it is slightly wider than the width of an opening in the or each slot, the opening being defined between the retaining members, such that the ramped portion must be pushed into the slot as far as the recess so that the mushroom shaped projection is prevented from being removed from its engagement with the retaining members.

The elongate sheath preferably comprises retaining tabs for further retaining the needle in the second position.

It is also preferred that the retaining tabs project from at least one side of the longitudinal channel, the retaining tabs being temporarily displaced to allow the needle to be received within the longitudinal channel when the elongate sheath is being pivoted to the second position.

The retaining tabs preferably project at an angle such as to prevent the needle being dislodged from the elongate sheath.

Still more preferably, the elongate sheath, when used to sheath the needle, encloses the needle completely from one side, including the tip.

According to a. second aspect of the invention, there is provided a safety needle assembly comprising the safety clip defined above connected to a collar at the base of a needle in a unitary construction.

According to a third aspect of the invention, there is provided a safety syringe assembly comprising the safety clip defined above connected to a hub of a syringe with a needle in a unitary construction.

### BRIEF DESCRIPTION OF THE DRAWINGS

Reference is now made to the drawings which depict:

FIG. 1 is a perspective view of a first embodiment of the safety clip of the present invention, the safety clip being shown with the attachment collar fully pivoted open about the hinge from the elongate sheath;

FIG. 2 is a longitudinal cross section of the safety clip as shown in FIG. 1, but upside down;

FIG. 3 is a longitudinal side view of the safety clip as shown in FIG. 1;

FIG. 4 is a longitudinal bottom view of the safety clip as shown in FIG. 1,

FIG. 5 is an end view from a pivoting end, at which the hinge is located, of the safety clip as shown in FIG. 1, but sideways;

FIG. 6 is a longitudinal top view, showing the longitudinal channel, of the safety clip as shown in FIG. 1;

FIG. 7 is an end view from a non pivoting end of the safety clip as shown in FIG. 1, but sideways;

FIG. 8 is a longitudinal top view, showing only a portion of the longitudinal channel, of a second embodiment of the safety clip of the present invention, the safety clip being shown with the attachment collar only about half pivoted open about the hinge from the elongate sheath;

FIG. 9 is a perspective view of the safety clip as shown in FIG. 8;

FIG. 10 is a different perspective view of the safety clip as shown in FIG. 8, and

FIG. 11 is a perspective view of the safety clip as shown in FIG. 8, the safety clip being shown with the attachment collar fully pivoted open about the hinge from the elongate sheath, and with the attachment collar securing the safety clip to a hub of a syringe so that the elongate sheath is in the first position in which a needle of the syringe is exposed and able to be used.

### BEST MODES FOR CARRYING OUT THE INVENTION

Prior art safety sheaths, clips and covers have suffered from several problems as set out in the background to the invention.

The present invention is adapted to be used with either a syringe which may have a detachable needle inserted therafter, or a needle alone, whereupon the assembly of needle and safety clip can be inserted onto the hub of a syringe. in alternate embodiments, the safety clip may form a portion of a combined syringe/clip/needle assembly or may be separate to a combined syringe/needle.

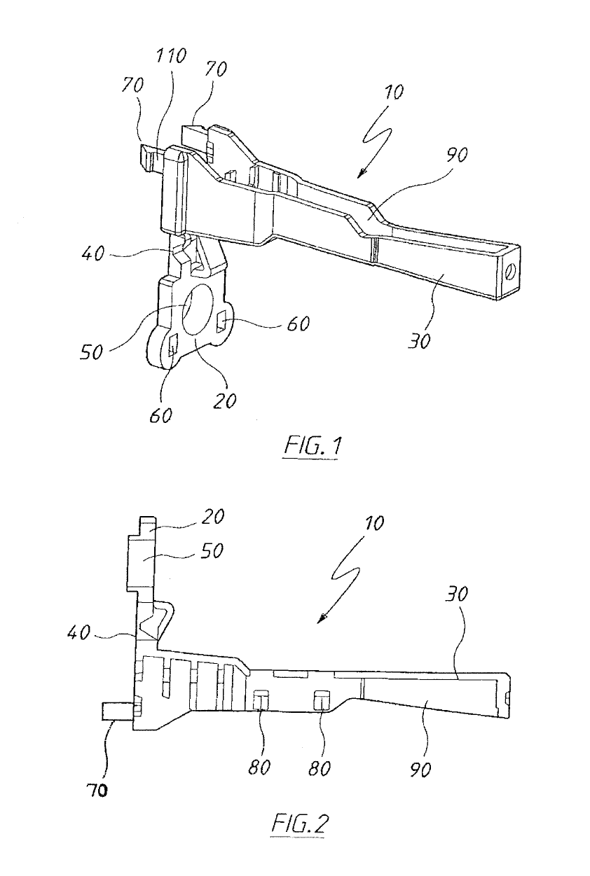

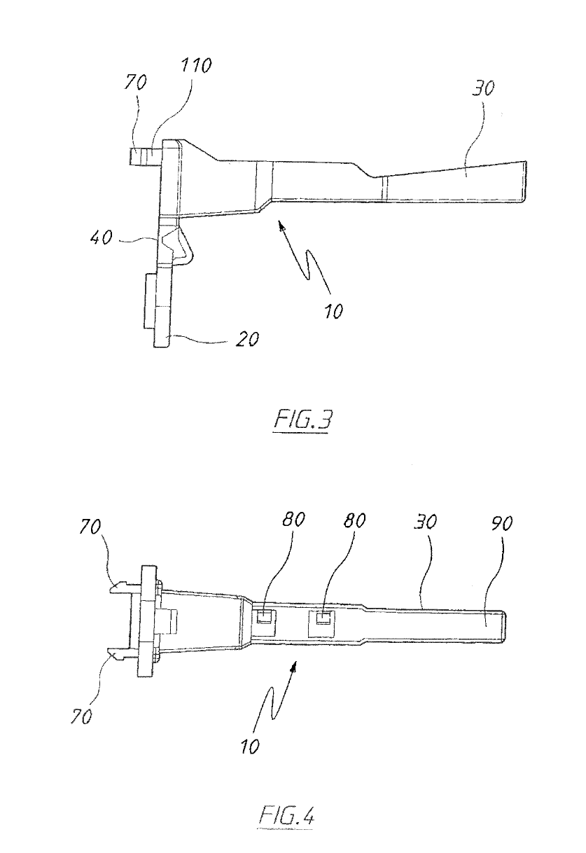

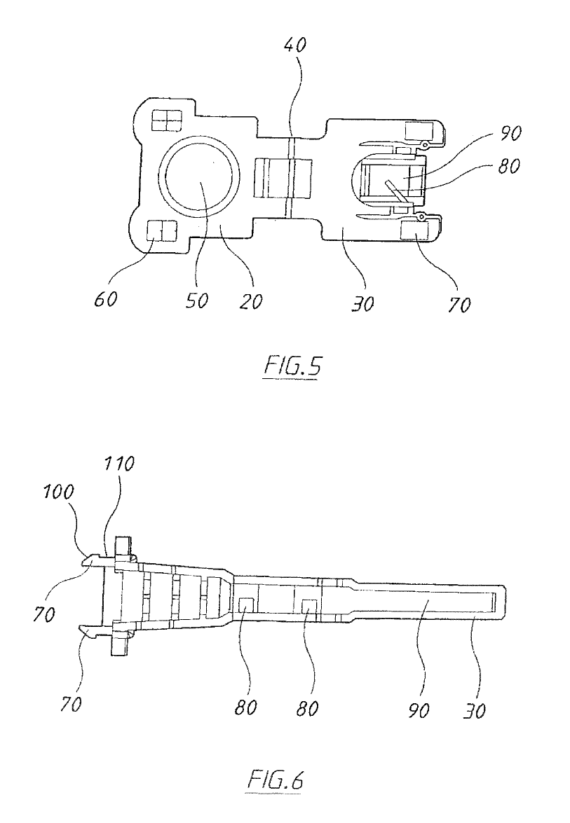

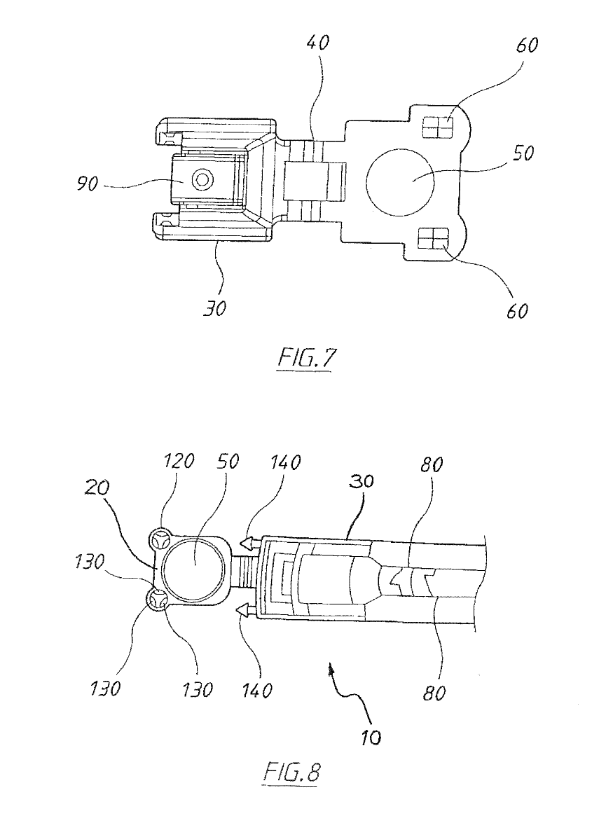

Referring to FIG. 1 there is depicted a safety clip 10 which is comprised of an attachment collar 20, elongate sheath 30 and hinge 40. The attachment collar 20 has a central aperture 50 through which either a hub of a syringe or the collar of the needle base is inserted and maintained in place through friction or alternatively via locking means. Attachment collar 20 is further comprised of slots 60 which engage with tabs 70 of the elongate sheath 30. The elongate sheath 30 also has a longitudinal channel 90 for receiving a needle.

In use, the safety clip 10 can be configured to be in one of two positions about the hinge 40. In a first position, the needle is exposed and the slots 60 are not engaged with tabs 70. The safety clip 10 can be configured to be in a second position by pivoting the elongate sheath 30 at the hinge 40 such that the channel 90 encases the needle and, with the application of pressure, causes the tabs 70 to be received or inserted into slots 60. Tabs 70 have a ramped portion 100 which, at its thickest point, is slightly wider than the width of the slot 60, such that the ramped portion 100 needs to be pushed into the slot where it is prevented from being removed from the slot 60 due to a recess 110.

The elongate sheath 30 also has retaining tabs 80 which retain the encased needle within the sheath. Retaining tabs 80 are formed as projections from one or either side of the channel 90 which are temporarily displaced when the needle is initially inserted into the channel 90, and thereafter are angled such that they prevent the needle from being dislodged out of the elongate sheath 30.

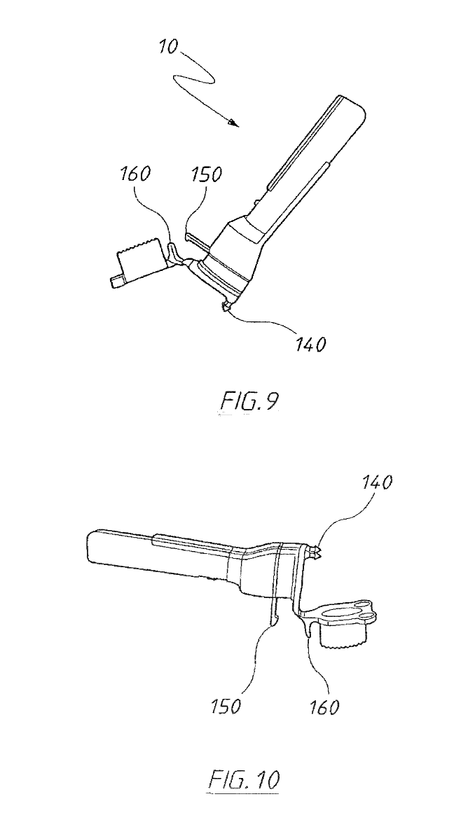

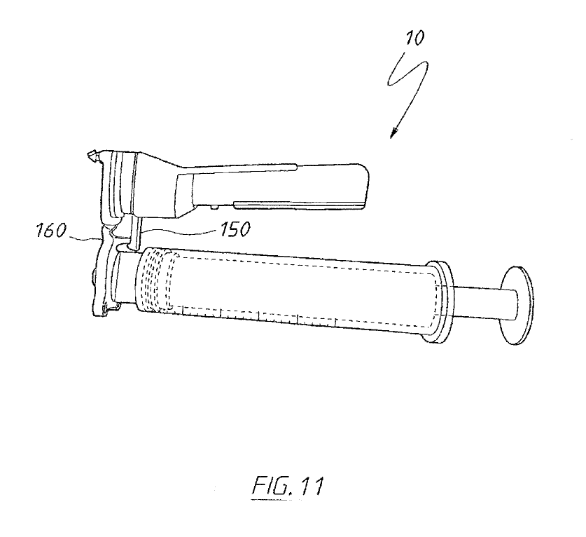

In an alternate embodiment, as shown in FIG. 8, the attachment collar 20 has slots or apertures 120 containing retaining members 130 which engage with mushroom shaped projections 140 which are located on the elongate sheath 30. The retaining members 130 are shaped to allow the mushroom shaped projections 140 to enter the apertures 120 and the shape of the mushroom shaped projections 140 prevent their removal from the apertures 120. This embodiment, as depicted in FIGS. 8 to 11, also depicts a second locking mechanism comprised of locking members 150 and 160 which operate to retain the safety clip 10 in the first or open position, where (as depicted in FIG. 11) the needle is not encased and the clip is retained against, in use, the barrel of the syringe, so as to prevent the clip from obscuring the view of the user of the syringe when giving an injection or removing bodily fluids. This second locking mechanism does not form a permanent lock, but rather a temporary fixing that can be released with minimum force.

The safety clip 10 of the present invention can be provided on its own so that it can be used with any standard syringes that have Luer locks or luer slip locks. Alternatively, the safety clip can be incorporated into a kit comprising a safety clip 10 and a syringe. In such case, the syringe and safety clip 10 can be made as a single component in a unitary construction. Alternatively, the safety clip 10 can be provided in a kit with a needle only. In such cases, the needle and safety clip 10 can be made as a single component in a unitary construction, and attached to any standard luer lock or luer slip lock syringe. In a further embodiment, the safety clip may be provided as an assembly of a safety clip and a combined (unitary) syringe and needle. In such cases, the safety clip may be incorporated into the construction of the combined (unitary) syringe and needle.

Various modifications will be apparent to the person skilled in the art where such variations are within the scope of the present invention.

### INDUSTRIAL APPLICABILITY

The present invention has industrial applicability in the fields of manufacturing and medical devices. Any description of the prior art is not to be taken as an indication or admission that it forms part of the common general knowledge in Australia or anywhere else.

1. A safety clip for a needle, the safety clip comprising:
an attachment collar having a central aperture through which can be secured either a hub of a syringe with the needle or a collar at the base of the needle,
an elongate sheath having a longitudinal channel along at least one side thereof for receiving the needle,
a hinge operably connected between the attachment collar and the elongate sheath,
wherein the attachment collar further comprises one or more slots containing retaining members, the slots being located at a remote end of the attachment collar, which remote end is furthest from an end of the attachment collar to which the hinge is connected,
and wherein the elongate sheath further comprises one or more mushroom shaped projections which extend from a remote end of the elongate sheath in a direction parallel to the longitudinal channel, and are insertable into the slots and engage with the retaining members,
such that the elongate sheath of the safety clip, when the safety clip is secured by its attachment collar to a hub of a syringe with the needle or to a collar at the base of the needle, can be pivoted about the hinge between a first position in which the needle is exposed and able to be used, and a second position in which at least a tip of the needle is sheathed in the longitudinal channel so that a user cannot be pricked by the needle,
and wherein the retaining members are shaped to prevent the mushroom shaped projections being removed from their engagement with the retaining members, thereby providing a permanent and secure locking between the slots and the mushroom shaped projections which prevents the elongate sheath being pivoted back to the first position when placed in the second position.

2. The safety clip of claim 1 wherein the or each slot contains three retaining members.

3. The safety clip of claim 1 wherein there are two slots and there are two mushroom shaped projections.

4. The safety clip of claim 1 wherein the or each mushroom shaped projection has a recess and a ramped portion which has a thickest point at which it is slightly wider than the width of an opening in the or each slot, the opening being defined between the retaining members, such that the ramped portion must be pushed into the slot as far as the recess so that the mushroom shaped projection is prevented from being removed from its engagement with the retaining members.

5. The safety clip of claim 1 wherein the elongate sheath comprises retaining tabs for further retaining the needle in the second position.

6. The safety clip of claim 5 wherein the retaining tabs project from at least one side of the longitudinal channel, the retaining tabs being temporarily displaced to allow the needle to be received within the longitudinal channel when the elongate sheath is being pivoted to the second position.

7. The safety clip of claim 6 wherein the retaining tabs project at an angle such as to prevent the needle being dislodged from the elongate sheath.

8. The safety clip of claim 1 wherein the elongate sheath, when in the second position, encloses the needle completely from one side.

9. A safety needle assembly comprising the safety clip of claim 1 connected to a collar at the base of a needle in a unitary construction.

10. A safety syringe assembly comprising the safety clip of claim 1 connected to a hub of a syringe with a needle in a unitary construction.
